(19) United States
(12) Patent Application Publication
(10) Pub. No.: US 2008/0299057 A1
(43) Pub. Date: Dec. 4, 2008

(54) Method and compositions for dispersing particulate solids in oil

(76) Inventor: Samuel Qcheng Sun Lin, Paramus, NJ (US)

Correspondence Address: Michael P. Aronson;MP&G Aronson Inc., 2 Mandarin Lane, West Nyack, NY, 10994, US

(21) Appl. No.: 12/154,890

(22) Filed: May 28, 2008

Related U.S. Application Data
(60) Provisional application No. 60/932,165, filed on May 29, 2007.

(51) Int. Cl. A61K 8/72; A61Q 17/04; A61Q 99/00

(52) U.S. Cl. 424/59; 424/78.31; 424/78.37

(57) ABSTRACT

This invention relates to a method for preparing dispersions of solid particles, especially pigments, metal oxides and mineral fillers in organic media, to dispersing compositions used in this method and to dispersions containing these dispersion compositions. The dispersing compositions include an oil soluble surfactant selected from the group consisting of phospholipids, alkyl acids, nonionic surfactants having an HLB less than 10, and mixtures thereof; either an anhydride based polymer selected from the group consisting of polyalkene succinic anhydrides; maleinized polyisoprene; maleinized polybutadiene; maleinized copolymers of isoprene and butadiene; copolymers of styrene and maleic anhydride; derivatives formed by reaction of the anhydride group of the anhydride based polymer with one or more of water, amines, alcohols, and polyols; and mixtures thereof; or a mixture of the said anhydride based polymer with polyhydroxystearic acid; and optionally a liquid oil carrier.

This application claims the benefit of U.S. Provisional Application No. 60/932,165 filed May 29, 2007.

### FIELD OF INVENTION

This invention relates to a method for preparing dispersions of solid particles, especially pigments, metal oxides and mineral fillers in organic media, to dispersing compositions used in this method and to dispersions containing these dispersion compositions. The dispersing compositions include specific oil soluble surfactants and anhydride based polymers.

### BACKGROUND OF INVENTION

A wide range of particles such as pigments, fillers and metal oxides are used for formulating coatings, plastics and compositions designed for topical application to skin. However, most, if not all, of these particles exhibit strong hydrophilic surface properties, are difficult to disperse in oils and are prone to wash off skin upon contact with high humidity, perspiration, and water. Furthermore the hydrophilic particles have a great tendency to form aggregates, thereby, not only losing their intended brightness of color, opacity and/or UV shielding power but also prevent in topical compositions the finished product from achieving consumer acceptable sensory properties.

Hydrophobic surface treatments have been used to alleviate the above problems. For example, U.S. Pat. No. 4,877,604 (1987) to Schlossman disclosed the surface treatment of pigments by chemical reactions with isopropyl titanium triisostearate. The treatment allowed higher levels of pigment to be incorporated into an anhydrous cosmetic composition and provided better sensory characteristics.

U.S. Pat. No. 5,366,660 (1994) to Tapley, U.S. Pat. No. 5,599,529 (1997) to Cowie, and U.S. Pat. No. 5,605,652 (1997) to Tapley disclosed the use of polyhydroxystearic acid as a sole dispersant for preparing oil dispersions of UV attenuated titanium dioxide and zinc Oxide particles.

U.S. Pat. No. 5,750,127 (1998) to Rokitowski disclosed an oil in water emulsion mascara formulation containing black iron oxide and a small amount of isostearic acid and polyhydroxystearic acid.

US 2002/0164297 (A1), to Ferrari, disclosed a cosmetic composition containing dispersion polymer particles and a pigment paste using polyhydroxystearic acid as a dispersant.

US 2008/0057008 A1 to Naden et al, U.S. Pat. No. 7,220,305B2 (2007), US 2003/0223940 A1 (2003) to Dransfield et al, and WO 2007/072008 A2 and WO 2007/034162 A1 to Kessell et al disclosed surface treatment of ultra fine titanium dioxide particles by coating with C₁₀-C₂₀ fatty acids in either water, solvent which is subsequently evaporated, or in a cosmetic vehicle in-situ. The references also disclose oil dispersions of coated titanium dioxide using polyhydroxystearic acid as a dispersant.

The polyhydroxystearic acid is polymerized from hydroxystearic acid and exists at room temperature as a viscous paste or as a gel which does not exhibit a pleasant sensory feel on the skin and is difficult to handle.

Common surface treatments for pigments are complicated and involve many steps such as milling with surface treatment chemicals, heating/mixing to carry out the chemical reaction between the chemicals and the particle surfaces, dry out solvents, grinding and jet milling of the resulting powders. This chemical reaction approach involves the use of dangerous solvents and/or high energy consumption, resulting in final surface treated powders which are many times more expensive than the untreated ones.

Special oil blends have also been practiced to alleviate this problem. CRODAPERSE is a patent-pending low energy dispersant system, commercially available from Croda Inc. at Edison, N.J. 08837, for dispersing pigments and metal oxide particles into the oil. According to its technical brochure—DS-193R-3, the said CRODAPERSE is a blend of three oils Caprylic/Capric triglyceride, Di-PPG-3 myristyl ether adipate, and sorbitan isostearate.

Pigment concentrates are concentrated pigment dispersions that are used as intermediates in the manufacture of coatings and plastics. They are often referred to as stainers, tinters, colorants, shading pastes, color master batches, etc. They offer manufacturers many benefits including: flexibility in “just-in-time” manufacturing, less inventory cost and space, less production time, less equipment cleaning time, etc. In general, the pigment concentrates should contain as high pigment levels as possible so that only relatively small amounts of the concentrates will be used in the final formulation.

U.S. Pat. No. 4,859,247 to Foye et al, disclosed a low viscosity, highly concentrated pigment dispersions by using an oil-soluble additive made by combining a long chain aliphatic amines having a molecular weight between 80 and 340 and a polybasic organic acid having a molecular weight between 50 and 250. The preferred pigment concentration was from 35% to 55%.

While studying the problem of dispersing hydrophilic metal oxides and pigments in various oils, I have discovered a convenient low energy method of dispersing hydrophilic pigments, metal oxides and fillers into various organic liquids. The method employs a dispersing composition which includes a mixture of specific oil soluble surfactants and anhydride based polymers. The method surprisingly allows the formation of pumpable and in many cases quite fluid concentrated dispersions, e.g., over 50 wt % solids, with a range of oils of different chemical composition. The dispersions are relatively easy to prepare following the methods disclosed herein. In addition, the dispersing compositions have sensory properties that are highly suited to cosmetic and personal care applications. The compositions can also be blended with various types of coating formulations and even plastics to allow simplified incorporation of particulate solids in these types of materials as well.

Because the dispersing compositions of the invention allows low cost and convenient processing by the methods described herein, the dispersing composition and the concentrated particulate dispersions so formed are advantageous intermediate for the manufacture of a range of products.

### SUMMARY OF THE INVENTION

One embodiment of the present invention is a method for preparing dispersions of particulate solids employing a specific dispersing composition. The method includes the step of combining particulate solids in powder form with a dispersing composition under suitable shear to form a uniform dispersion, said dispersing composition comprising:
a. 10% to 90% by weight of total composition of an oil soluble surfactant selected from the group consisting of phospholipids, alkyl acids, nonionic surfactants having an HLB less than 10, and mixtures thereof;
b. 10% to 90% by weight of total composition of either an anhydride based polymer selected from the group consisting of polyalkene succinic anhydrides; maleinized polyisoprene; maleinized polybutadiene; maleinized copolymers of isoprene and butadiene; copolymers of styrene and maleic anhydride; derivatives formed by reaction of the anhydride group of the anhydride based polymer with one or more of water, amines, alcohols, or polyols; and mixtures thereof; or a mixture of the said anhydride based polymer with polyhydroxystearic acid; and
c. 0 to 50% by weight of composition of a liquid oil carrier.

Another embodiment of the invention is a dispersing composition described above which can be prepared separately and used as required to prepare various dispersions such as dispersions of pigments, metal oxides or mineral fillers and other amorphous or crystalline solids. The dispersing compositions of the invention not only provide a route to simple and convenient manufacture but also provide tangible improvement in products designed to be applied to skin, i.e., topical products such as health, beauty, cosmetic and other personal care products. These benefits include improved in-use tactile properties, improved coverage, durability and rub-out on the skin.

Another embodiment is a concentrated dispersion in which pigments, fillers and/or metal oxide particles are dispersed in the composition. Such dispersions can be used as convenient intermediates for the manufacture of a range of products which contain particulate solids.

A still further embodiment is a topical skin care composition that includes by weight of composition: 0.04% to 1.5% of the dispersing composition; 1% to 25% of pigments, fillers, and metal oxides; and one or more ingredients selected from the group consisting of cosmetic oils, water, active ingredients, benefit agents, and various auxiliary ingredients.

These and other variations, applications and advantages of the method and articles of manufacture described herein will become clear from the description of the invention which follows.

### BRIEF DESCRIPTION OF THE DRAWING

FIG. 1 is a graph comparing the viscosities of concentrated dispersions as a function of the dispersed solids content of TiO₂ as described in Example 2

FIG. 2 is a graph comparing the viscosities of concentrated dispersions as a function of dispersed solids content of iron oxide as described in Example 3.

FIG. 3 is a graph comparing the viscosities of concentrated dispersions as a function of dispersed solids content of TiO₂ as described in Example 4.

### DETAILED DESCRIPTION OF THE INVENTION

As used herein % or wt % refers to percent by weight of an ingredient as compared to the total weight of the composition or component that is being discussed.

Except in the operating and comparative examples, or where otherwise explicitly indicated, all numbers in this description indicating amounts of material or conditions of reaction, physical properties of materials and/or use are to be understood as modified by the word “about.” All amounts are by weight of the final composition, unless otherwise specified.

For the avoidance of doubt the word “comprising” is intended to mean “including” but not necessarily “consisting of” or “composed of.” In other words, the listed steps or options need not be exhaustive.

The invention in its first embodiment relates to a method for dispersing pigments, fillers, metal oxides, other crystalline or amorphous particles and their mixtures in oils for various applications including coatings, plastics and topical products. The term topical products is used to encompass products that are applied topically to the skin including: health products such as sun screens and anti-acne treatments, beauty products such as color cosmetics and skin lightening treatments, and personal care products such as moisturizing and anti-aging creams and lotions. The topical products can be in various forms such as lotions, creams, gels, ointments and even sprays and wipes.

The method broadly involves the combining of particulate materials with a dispersing composition that includes specific oil soluble surfactants and anhydride based polymers under sufficient shear to allow uniform mixtures to form.

In several embodiments of the instant invention, the method is used to prepare either intermediates or end use topical products. For these topical applications, the ingredients used in the method, i.e., the dispersing composition, particulate materials and additives must be dermatologically acceptable. The term “dermatologically acceptable” means that the dispersing compositions and particulates, e.g., metal oxides, must be non-irritating and biologically safe for prolonged contact with skin. For example, it must pass standard safety skin irritation tests such as repeated insult patch test (RIPT), and it must contain less than 20 ppm of lead, 3 ppm of arsenic, 15 ppm of cadmium, and 1 ppm of mercury.

Other applications are more industrial in nature and produce products in which the ingredients are either fixed, e.g., present in cured polymer coatings and plastics, and/or do not come into prolonged contact with skin. Here dermatological acceptability of all the ingredients and the composition as a whole is less important or not important.

Another embodiment of the invention is the dispersing composition itself which is a useful intermediate or processing aide useful in the preparation of products that contain particulates in highly dispersed form, e.g., having a particle size less than 10μ, preferably less than 1μ, and most preferably 0.2μ or less.

Another embodiment of the invention is a concentrated dispersion in which pigment, fillers and/or metal oxides in particulate form are dispersed in a composition formed from the dispersing composition of the invention and optional carrier oils (the terms “carrier oil”, “liquid oil” and “liquid carrier oil” are used interchangeably). These concentrated dispersions are useful as intermediates for the preparation of color coating compositions, plastics and various topical compositions.

Another embodiment of the instant invention are topical products that contain particulate solids in compositions that include the dispersing composition described herein. The particulate solids are metal oxide (especially those which function as UV screens) pigments, fillers and mixtures of these particulate materials.

These and additional embodiments are described in detail below.

Anhydrous Dispersing Compositions

The composition which is suitable for dispersing metal oxides and pigments and other particulate materials includes oil soluble surfactants, polymers and optional liquid oil carriers.

The oil soluble surfactant is selected from the group consisting of phospholipids, alkyl acids, and nonionic surfactants having an HLB less than 10 and mixtures thereof.

By the term “oil soluble surfactant” is meant a surfactant that contains about 14 to 40 carbon atoms in the molecule and has a relatively lesser amount of water solubilizing groups so that the surfactant tends to concentrate and preferably dissolve in an oil phase rather than in a water phase.

The HLB value, or hydrophilic-lipophilic-balance value, of a surfactant is a well known term to those skilled in the art. The HLB value is related to the solubility of the surfactant, wherein a surfactant with a low HLB value, e.g., about 10 or less, tends to be oil soluble and a surfactant with a high HLB value, e.g., greater than about 10, tends to be water soluble. Surfactants useful in the current invention have an HLB value 10 or less, preferably 6 or less.

Preferably, the oil soluble surfactant has a solubility of at least about 0.1 g per 100 ml in oils commonly used in cosmetic applications, e.g., triglyceride oils or volatile silicones, to form a true solution.

The oil soluble surfactant is present at a level of 10% to 90% by weight of the dispersing composition, preferably at a level of 10% to 60% and most preferably at a level of 25% to 45%.

Phospholipids are suitable oil soluble surfactants. There are two classes of phospholipids: those that have a glycerol backbone and those that contain sphingosine. Phospholipids that contain a glycerol backbone are called phosphoglycerides (or glycerophospholipids), which are the most abundant class of phospholipid found in nature. The most abundant types of naturally occurring phosphoglyceride are phosphatidylcholine, phosphatidylethanolamine, phosphatidylserine, phosphatidylinositol, phosphatidylglycerol, and cardiolipin.

The most suitable phospholipids for purposes of the current invention are natural or synthetic phosphoglycerides and include, but not limited to, phosphatidylcholine, phosphatidylethanolamine, phosphatidylinositol, and phosphatidic acid. A preferred phospholipids is phosphatidyicholine which is commonly known as lecitihin.

Oil soluble alkyl acids are broadly defined herein as carboxylic acids and phosphoric acids and their salts which contain an alkyl chain having at least 14 carbon atoms, preferably at least 16 carbon atoms and most preferably at least 18 carbon atoms. The alkyl chain can be branched or unbranched, saturated or unsaturated. When salts are used, particularly suitable cations are those formed with an organic base such as monoethanolamine and triethanolamine.

Examples of suitable oil soluble alkyl carboxylic acids includes isostearic acid, isostearyl sebacate, oleic acid, hydroxystearic acid, neodecanoic acid, butyloctanoic acid, stearic acid, hexyldecanoic acid, oleth-3 carboxylic acid, stearic acid, and glyceryl stearate citrate.

Suitable oil soluble alkyl carboxylic acids also can include alkoxy groups in the molecule. For example, the surfactant can comprise one or more ethylene oxide, propylene oxide, butylenes oxide, or mixtures of the two, e.g., an alkyl ethoxy carboxylic acid, as long as the surfactant is oil soluble.

Dimer acid and trimer acid are still other types of suitable oil soluble alkyl carboxylic acid. These surfactants are dimers and trimers of unsaturated fatty acids such as linoleic acid and oleic acid and contain 36 carbons and 48 carbons respectively. They are commercially available as EMPOL 1008 from Emery Industry, and UNIDYME from Arizona Chemicals.

Suitable alkyl phosphoric acids and their salts can also include alkoxyl groups in the molecule. For example, the surfactant can comprise one or more ethylene oxide groups, one or more propylene oxide groups or a mixtures of these groups, e.g., an alkyl ethoxy phosphate, as long as the surfactant is oil soluble.

Examples of suitable oil soluble alkyl phosphoric acids include cetyl phosphate, stearyl phosphate, ceteth-3 phosphate, trilaureth-4 phosphate, triceteareth-4 phosphate and oleth-3-phosphate. Although the surfactants are designated as “phosphates in CTFA nomenclature, the materials are phosphoric acid derivatives, e.g., fatty alcohol monoesters of phosphoric acid. Di-ester phosphates (two alkyl groups) are also suitable but less preferred.

Another suitable class of oil soluble surfactant is non-ionic surfactants having a HLB less than 10, preferably less than 8 and most preferably less than 6. Examples include: sorbitan fatty acid ester such as sorbitan monostearate, sorbitan monooleate and sorbitan monolaurate; sucrose fatty acid esters such as sucrose oleate and palmitate; alkyl glycoside, and polyglycerol esters such as polyglyceryl-2 dipolyhydroxystearate, polyglyceryl polyricinoleate and decaglycerol decaoleate.

Polymers suitable for the particulate dispersing compositions are a particular group of anhydride based polymers. These polymers have one or more pendant anhydride groups, or pendent groups derived (formed) by the reaction of the anhydride with water (to form carboxylic acid), alcohols and polyols (to form esters), and amines to form amides.

In one suitable embodiment, the polymer is an anhydride based polymer selected from the group consisting polyalkene succinic anhydrides; maleinized polyisoprene; maleinized polybutadiene; maleinized copolymers of isoprene and butadiene; copolymers of styrene and maleic anhydride; derivatives formed by reaction of the anhydride group of the anhydride based polymer with one or more of water, amines, alcohols or polyols; and mixtures thereof (i.e., mixtures of the above classes of anhydride based polymers).

These polymers have functional groups comprising anhydrides and/or their derivative carboxylic acid, ester and/or amide groups derived from the reaction of an anhydride with water, an alcohols and/or amine. The polymer can comprise a mixture of these functional groups, e.g., mixtures of acid and ester functionality. Preferably, the polymer should have on average less than ten anhydride groups or anhydride equivalent groups (i.e., anhydride groups reacted with water, alcohols, polyols and/or amines) per polymer molecule and preferably, less than five anhydrides or anhydride equivalent groups per molecule.

For topical product applications, the anhydride based polymers must be dermatologically acceptable in the sense defined above, and thus should be substantially free of the harmful heavy metals. In this case the polymers should contain less than the following threshold levels of metal impurities: 20 ppm of lead, 3 ppm of arsenic, 15 ppm of cadmium, and 1 ppm of mercury.

The polymer should have a molecular weight of 1,000 to 50,000 Daltons, preferably 1,000 to 30,000 Daltons and most preferably have molecular weight of 1,000 to 10,000 Daltons.

Polyalkene succinic anhydride is a reaction adduct of maleic anhydride with a polyalkene which is a polymer of olefin monomers such as isobutene, butene, propylene, 1-hexene, ethylene, and 1-pentene. Under certain reaction conditions the maleic anhydride reacts with residual terminal double bonds on the polyolefin to graft succinic anhydride groups at the ends of the polymer chain. Their structures and method of preparation are disclosed in the prior art, for examples: U.S. Pat. No. 3,163,603 to Le Suer; U.S. Pat. No. 3,231,587 to Rense et al; U.S. Pat. No. 4,234,435 to Meinhardt et al; and U.S. Pat. No. 7,339,007 to Irving et al, incorporated by reference herein.

The preferred polymer is polyisobutene succinic anhydride with molecular weight less than 5000 Daltons. An example is the product PCA 9050 from Soltex in Houston, Tex.

Derivatized polyalkene succinic anhydride are also suitable. These polymers contain carboxylic acid, ester and amide functional groups that are formed by the reaction of the anhydride with for example water, alcohol, polyhydroxyl compounds and amines. Examples of polyhydroxyl compounds include, polyglyceryl, sorbitol, and glycerine.

Other suitable polymers are “maleinized” polymers. These polymers are the reaction products formed by reaction of polydienes such as polybutadiene, polyisoprene, or copolymers of butadiene and isoprene with maleic anhydride. The reaction product is a polymer with pendant succinic anhydride groups not only at terminal positions but distributed throughout the polymer chain. These polymers are commercially available as RICON resins from Sartomer located at Exton, Pa., USA and LIR and LBR polymers from Kuraray located in Tokyo, Japan. Also useful are their derivatives formed by reacting the anhydride groups with water, alcohols, polyhydroxyls and amines (including polyamines), and mixtures thereof.

Another suitable class of polymer is copolymers of styrene and maleic anhydride as well as their ester and amide derivatives with water, alcohols and amines. These polymers are known as SMA resins, and commercially available from Sartomer located in Exton, Pa., USA, as SMA 1000, 3000, 31890, 2625 with molecular weight ranging from 2000 to 10,000.

In a second embodiment, the polymer is a mixture of the one or more anhydride based polymers as selected and described above with polyhydroxystearic acid. Polyhydroxystearic acid is a polyester which is obtainable for example by heating hydroxystearic acid, eg. commercial 12-hydroxystearic acid or else 9- or 10-hydroxystearic acid, at from 160° to 200 degree C. in the presence of an inert organic diluent, eg. toluene or xylene, and removing from the reaction mixture the water formed in the course of the esterification reaction. Commercial examples of polyhydroxystearic acid include SOLPERSE resins by Lubrizol at Cleveland, Ohio, and PELEMOL PHS-8 by Phoenix Chemicals at Somerville, N.J.

The composition contains 10% to 90% by weight of the polymer or polymers described above, i.e., anhydride based polymer(s) alone or in combination with polyhydroxystearic acid, preferably 10% to 60% and most preferably of 25% to 45% by weight.

When the oil soluble surfactants are restricted to only phospholipids, dimer acids and trimer acids or their mixtures, the dispersing polymer can be polyhydroxystearic acid and can be employed as the sole dispersing polymer in an alternative though less preferred embodiment of the dispersing composition.

Here the composition comprises

a) oil soluble surfactants selected from synthetic or natural phospholipids, dimer acid, and trimer acids present in the composition at a level of 10% to 90% by weight of the composition, preferably at a level of 10% to 60% and most preferably at a level of 25% to 45%,

b) polyhydroxy stearic acid present in the composition in the amount of 10% to 90% by weight of any composition, preferably 10% to 60% and most preferably of 25% to 45% by weight of composition, and

c) optionally liquid oil carrier as described below present at a level of up to about 50% by weight of composition.

A useful optional ingredient in the anhydrous dispersing composition is a liquid oil which is used as diluents or carriers to extend the composition and to control viscosity especially when pigments, fillers, metal oxide or other particulate material is incorporated to form concentrated dispersions.

Suitable liquid oil carriers include natural oils, synthetic esters (linear, branched, or cylic), mineral oils, hydrocarbons oils, silicone oils, ketones having more than 6 carbons, ethers having more than 6 carbons, monoalcohols having at least 6 carbons, etc. Silicone oils including volatile silicones oils, organo-modified silicone oils, and mixtures thereof. Examples of natural oils include jojoba oil, sunflower oil, avocado oil. Examples of synthetic ester oils include isononyl isononoate, C₁₂₋₁₅ alkyl benzoate, propylene glycol dibenzoate, isopropyl palmitate, lauryl lactate, triethylhexyl citrate, caprylic/capric triglyceride, neopentyl glycol dicaprylate/dicaprate, and ethylhexyl palmitate. Examples of hydrocarbon oils include isoparaffin, isohexadecane, isododecane, polybutene, polyalphaolefin and mineral oil. Examples for the fluorinated oils are FOMBLIN from Montefluos, SILUBE GME-F from Siltech.

Suitable liquid oil carriers as listed above are characterized by having an overall Hansen solubility parameter of less than or equal to 22 (MPa)^{1/2}, preferable less than 20 (MPa)^{1/2}, and most preferably less than 18 (MPa)^{1/2}.

The Hansen solubility is defined in the article of “Solubility parameter values” by Eric A Grulke in “Polymer Handbook”, 3^{rd} edition, Chapter VII, pages 519-559, and in the articles of “The three-dimensional solubility parameters: by C. M. Hansen, in “J. Paint Technol., 39, 105 (1967).

When the composition is intended for topical applications, the liquid oil carrier must be dermatologically acceptable.

In some instances, it is preferable that the dispersing composition does not contain added water, i.e. the composition is preferably substantially anhydrous. By substantially anhydrous is meant that the level of water in the dispersing composition is less than 5% by weight, preferably less than 2% and more prefer less than 1% and most preferably is less than 0.1% or absent.

Concentrated Dispersions

Another embodiment of the invention is a concentrated dispersion in which pigment, fillers and/or metal oxide particles are dispersed in the dispersing composition described above. Such compositions provide pre-dispersed easy-to-use intermediate for the manufacture of coatings, plastics, and various topical compositions.

Product manufacturers increasingly prefer liquid ingredients and intermediates that are pumpable and easy to transfer. It has unexpectedly been discovered that the liquid dispersing compositions described herein can readily incorporate particulate materials such as pigments and metal oxides to form concentrated dispersions that have relatively low viscosity and are very easy to handle. Furthermore, the said dispersing compositions are capable of dispersing non-surface treated pigments, metal oxide and filler particles easily into oils without the need of costly surface treatments.

The concentrated dispersion of the present invention comprises 50% to 80% by weight of particulate materials (e.g., pigments, fillers, and metal oxide UV shielding particles), 3% to 20% by weight of the dispersant blend of this invention based on the weight of the particulate material (i.e., 3-20 gm dispersing composition per 100 gm of particulates (e.g., pigment)), and a liquid oil carrier.

One of the advantageous properties of the concentrated dispersions described herein is their ease of handling as an intermediate, when the levels of oil soluble surfactants, dispersing polymers and carrier liquid, are chosen so that the composition remains relatively fluid and pumpable. Specifically, the viscosity of the composition, which contains at least 50% particulate material, more preferably at least 60% particulate material should preferably be less than 500,000 cP, more preferably less than 300,000 cP, still more preferably less than 200,000 cP and most more preferably less than 50,000 cP measured by the method described below.

A preferred composition would include:

i) 3% to 20% of a dispersing composition based on the weight of the pigments filler or metal oxide particles being dispersed comprising:
a. 10% to 90% by weight of dispersing composition of an oil soluble surfactant selected from the group consisting of phospholipids, alkyl acids, nonionic surfactants having an HLB less than 10, and mixtures thereof;
b. 10% to 90% by weight of total composition of either an anhydride based polymer selected from the group consisting of polyalkene succinic anhydrides; maleinized polyisoprene; maleinized polybutadiene; maleinized copolymers of isoprene and butadiene; copolymers of styrene and maleic anhydride; derivatives formed by reaction of the anhydride group of the anhydride based polymer with one or more of water, amines, alcohols or and polyols; and mixtures thereof; or a mixture of the said anhydride based polymer with polyhydroxystearic acid;

ii) 50% to 80% by weight of pigments, fillers, metal oxides or combinations thereof in particulate form; and

iii) a liquid oil;

Especially preferred are compositions in which the oil soluble surfactant, anhydride based polymers, and liquid oil carriers are selected such that the viscosity of the dispersing composition is less than 20,000 cP, preferably less than 10,000 cP, and most preferably less than 3,000 cP measured by the method described below.

When the concentrated dispersion is intended as an intermediate for topical applications, the dispersing composition and the pigments, metal oxides, fillers and additional optional ingredients must be dermatologically acceptable ingredients.

Suitable pigments include titanium dioxide, zinc oxide, iron oxides and iron hydroxide of red, black, brown, ultramarine blue, and yellow colors, organic lakes, etc. which are substantially free of heavy metals such as lead, arsenic, cadmium, mercury, and the like.

Fillers include mica, talc, sericite, calcium carbonate, kaolin, and silica.

A concentrated dispersion that is particularly suitable as an intermediate for topical sunscreen composition contains metal oxide which are effective as UV screens. Preferred metal oxide particles suitable as UV shielding particles include: titanium oxide, zinc oxide, cerium oxide and mixtures thereof. Ultra fine titanium dioxides are commercially available from a number of suppliers. For example, UV TITAN from Kemira is a series of rutile titanium dioxides, including UV TITAN M111, M160, M170, M262, M263, and M610. Titanium dioxide P25S and T80S from Degussa are anatase titanium dioxides. TTO series are rutile titanium dioxides from Ishihara Corporation USA (ISK), including TTO S-6, TTO S-4, MPT-404, TTO-V3 & V4, etc. MT series micro fine titanium dioxides are from Tayca. Ultra fine zinc oxides are commercially available from BASF as Z-coate®, Z-coate® HP-1, and Z-coate® Max, from Sumitomo Osaka Cement as ZnO-350, etc., and from other suppliers.

Concentrated oil dispersion of solids in particulate form can be conveniently prepared in the following batch process:

i) The oil soluble surfactant and polymer dispersants, a suitable carrier oil and other optional ingredients are added to a mixing vessel. These ingredients can be added individually or preferably as a complete composition. Furthermore, none, only part of or all of the liquid carrier and optional ingredients can be added at this stage or at other stages in the process.

ii) The pigment, metal oxide powder or filler is then gradually added to the vessel while mixing vigorously with a Cowles disperser or other high shear mixing equipment at a temperature ranging from room temperature to 100° C. When the dispersions reach a desired viscosity and is in a homogenous state, the mixing is stopped, and composition transferred to a container.

Suitable alternative high-shear equipments include colloid mills, high-pressure homogenizers, three-roll mills, ball mill, bead mill, etc. Examples of commercial high shear equipments include Gaulin homogenizer, micro fluidizer, DeBee 2000P, Eppenbach colloid mill and the like.

Alternatively, the concentrated dispersion can be prepared in a continuous process by combining the dispersing composition, particulate solids and optional ingredients via appropriate liquids and solids feeders and passage through in-line mixers and a single or multistage colloid mill.

Another embodiment of the instant invention is topical products that contain particulate solids in combination with the dispersing composition described herein. Especially suitable particulate solids are metal oxide. UV screens, pigments, fillers and mixtures of these particulate materials. The topical products can be emulsions of oil and water.

Such topical compositions include the following dermatalogically acceptable components:

i) 0.04% to 2% by weight of topical composition, preferably 0.08% to 1.6%, and most preferably 0.15% to 0.6%, of the anhydrous dispersing composition that includes: a) 10% to 90% by weight of dispersing composition of an oil soluble surfactant selected from the group consisting of phospholipids, alkyl acids, nonionic surfactants having an HLB less than 10, and mixtures thereof; 10% to 90% by weight of total composition of either an anhydride based polymer selected from the group consisting of polyalkene succinic anhydrides; maleinized polyisoprene; maleinized polybutadiene; maleinized copolymers of isoprene and butadiene; copolymers of styrene and maleic anhydride; derivatives formed by reaction of the anhydride group of the anhydride based polymer with one or more of water, amines, alcohols or polyols; and mixtures thereof; or a mixture of the said anhydride based polymer with polyhydroxystearic acid; and c) 0 to 50% by weight of dispersing composition of a liquid oil carrier;

ii) 1% to 35% by weight of topical composition, preferably 2% to 20% and more preferably 5% to 15% and most preferably 5% to 10% of a dermatologically acceptable metal oxide, pigment, filler or mixtures thereof; and

iii) one or more ingredients selected from the group consisting of additional liquid oil carrier(s), active ingredients, benefit agents, and auxiliary ingredients.

Active ingredients and benefit agents include but are not limited to skin moisturizers, skin whitening agents, vitamins, anti-oxidants, blood clotting agents, wound healing agents, self tanning agents, anti-acne agents, absorbents for wounds, antibacterial, hair conditioner, sebum control agents, anti-aging agents, depilatory agents, antiperspirants and organic sunscreens. Functional and auxiliary ingredients include, but are not limited to, water, emulsifiers, emollients, fragrance, colorants, preservatives, humectants such as polyols, and other suitable carriers including water.

### EXAMPLES

The following examples are shown as illustrations of the invention and are not intended in any way to limit its scope.

### Example 1

Table 1 illustrates various anhydrous dispersing compositions according to the invention. The compositions are prepared by mixing all components in a beaker with an overhead mixer initially at room temperature to a temperature of 100° C.

TABLE 1
Exemplary anhydrous dispersing compositions

| | Dispersing | | | | | | | | | | |
|---|---|---|---|---|---|---|---|---|---|---|---|
| | Composition No. | | | | | | | | | | |
| | 1 | 2 | 3 | 4 | 5 | 6 | 7 | 8 | 9 | 10 | 11 |
| | Weight % | | | | | | | | | | |
| Polyhydroxystearic | 25 | 35 | 10 | | | 50 | | | 5 | 45 | |
| acid | | | | | | | | | | | |
| Styrene/maleic | 8 | | 1 | 4 | 15 | | | | 1 | | |
| anhydride* | | | | | | | | | | | |
| Maleinized | 8 | 7% | 10 | 50 | 15 | | | 20 | 10 | | |
| Polybutadiene# | | | | | | | | | | | |
| +Polyisobutene | | | | | | | 75 | 40 | 40 | | 90 |
| succinic anhydride | | | | | | | | | | | |
| lecithin | | | | | | | | 5 | 5 | 15 | 5 |
| Isostearic acid | 20 | 12 | | | 10 | | 10 | | | | 5 |
| Dimer acid | | | 20 | 10 | 5 | 25 | | | 3 | 5 | |
| Oleth-3 phosphate | 4 | 4 | 10 | 5 | | | | | | | |
| Sorbitan oleate | 2 | 2 | 2 | | | | | | 2 | | |
| Glyceryl stearate | | | | 3 | | | | | | | |
| Citrate | | | | | | | | | | | |
| Methyl glucose | | | | 3 | | | | | | | |
| isotearate | | | | | | | | | | | |
| Octyl Palmitate | 35 | | | | | 25 | | | | 40 | |
| Caprylic/Capric | | 40 | 47 | | | | 15 | 25 | 34 | | |
| Triglyceride | | | | | | | | | | | |
| Soya Oil | | | | 25 | 55 | | | | | | |
| total | 100 | 100 | 100 | 100 | 100 | 100 | 100 | 100 | 100 | 100 | 100 |

*SMA 31890.
#Ricon 131 M5.
+PCA 9050 by Soltex Inc.

### Example 2

This study compares the dispersing power of a dispersing composition used according to the method of the present invention, specifically Composition 2 (Table 1) with a commercial dispersing oil CRODAPERSE low energy disperse system.

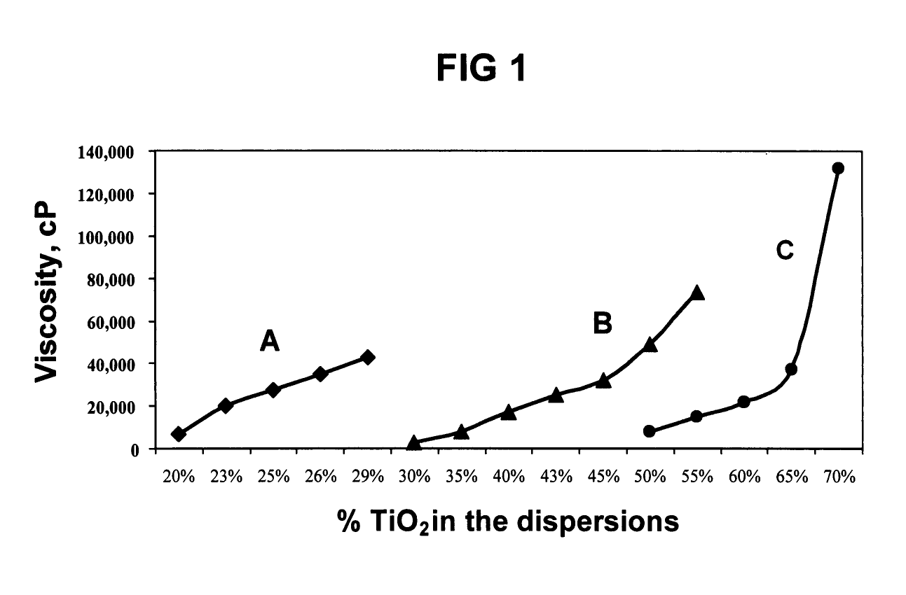

Non-surface treated white titanium dioxide pigment was added into CRODAPERSE oil incrementally and mixed using a COWLES disperser to prepare dispersions having different pigment concentration. Similarly, two other series of dispersions were prepared. One dispersion contained the titanium dioxide pigment in just a carrier oil composed of caprylic/capric triglycerides (CCT) and one set of dispersions made with titanium dioxide in combination with the dispersing composition 2 of Table 1 and caprylic/capric triglyceride oil. The weight % of the dispersing composition 2 relative to pigment was kept at 4% (i.e., 4 gm of composition 2 per 100 gm of TiO₂). Composition 2 mixed easily with the caprylic/capric triglyceride oil at room temperature. The viscosities at 1 rpm were measured and plotted against the wt % of titanium dioxide in the dispersion with the results given in FIG. 1.

The viscosity was measured at 5 rpm with a Brookfield Viscometer fitted with regular RV spindles # 2, 3, 4, or 5. Since the dispersions exhibited shear-thinning rheology, their viscosities were also measured over a range of shear rates.

The more effective the dispersing composition is in compatibilizing the pigment in the medium, the lower is the viscosity of the dispersion at any given pigment concentration. With reference to FIG. 1, to reach a viscosity of 40,000 cP only required about 29% pigment in CCT (curve A) and about 50% pigment in CRODAPERSE (curve B), but required about 65% pigment using the dispersing composition No. 2/CCT mixture (curve C). These results demonstrate the significantly greater dispersing power of the dispersing compositions of the invention.

Furthermore, at 65% pigment concentration, only about 2.6% weight of composition 2 was required in the total dispersion, compared to the 50% CRODAPERSE oil. The concentrated dispersions made with composition No. 2 was flowable, easily rubbed out when applied to skin and exhibited a very pleasant emollient feel on the skin. The compositions and method of the invention provide improvements in terms of ease of manufacture and handling of concentrated dispersions, efficient manufacture, and provided intermediates having excellent sensory properties.

### Example 3

This study compared the dispersion of black iron oxide pigments, which had undergone complicated surface chemical treatment with octyltriethoxysilane, and the dispersion with of non-surface-treated iron oxide pigment but using dispersing composition No. 2 blended with caprylic/capric triglycerides as the dispersing medium. The concentration of dispersing composition was 8% by weight relative to the weight of the pigment. The non-surface treated black iron oxide pigment in this case was iron oxide C33134 obtained from Sun Chemicals, while the surface treated pigment was Unipure black LC 989 ASEM, commercially available from Sensient Technologies Inc. at Milwaukee, Wis.

Dispersions with CCT solvent were prepared as in Example 2.

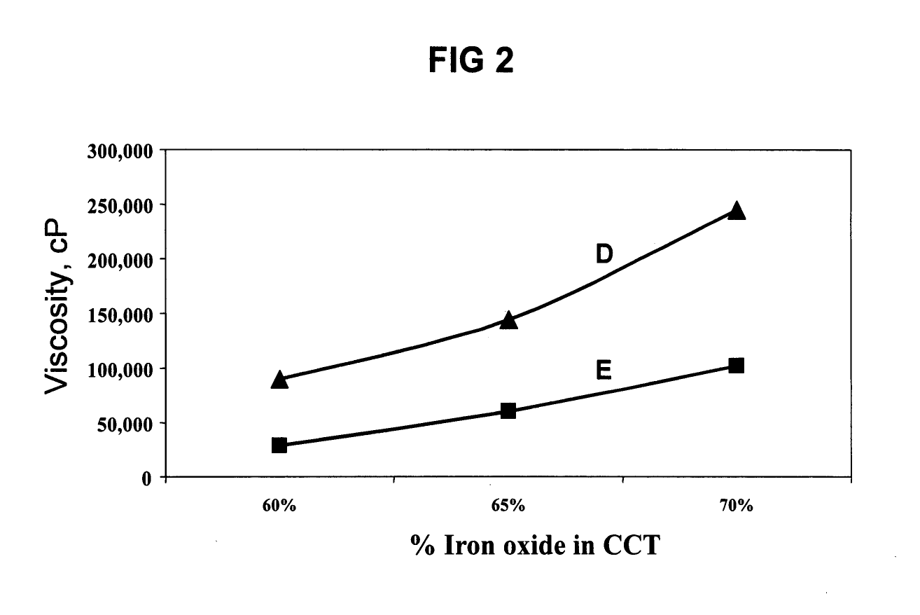

The results are presented in FIG. 2. Curve D is the viscosities of dispersions of the surface treated pigment dispersed in CCT while curve E corresponds to the viscosities of dispersions of the untreated pigment in the same dispersion medium containing dispersing composition 2.

The results in FIG. 2 indicates that the viscosity of the bare black iron oxide dispersion (curve E) using composition 2 at all iron oxide concentration was in fact lower than that of the special surface treated black iron oxide pigment (curve E). This result was unexpected since the iron oxide pigment with special chemical reaction treatment was designed to disperse easily into the oils and thus expected to provide lower viscosity dispersion than untreated pigments. The result demonstrates the superior dispersing powder of the dispersing compositions of the invention and the ease of use of the inventive method.

### Example 4

This study is similar to the one described in Example 3 and compares the dispersion of another pigment, which has undergone complicated surface chemical treatments, and a dispersion made with virgin non-surface-treated pigment (R56379, obtained from Whittaker, Clark & Daniel Inc) but using the dispersing composition No. 2 and No. 11 (Table 1) of the present invention at 4% and 10% by weight of pigment, respectively. BTD-401 titanium dioxide pigment from Kobo Product Inc was used. This pigment had been treated with isopropyl titanium triisostearate as disclosed in the U.S. Pat. No. 4,877,604 (1987) to facilitate its dispersion into cosmetic oils.

Dispersions were prepared as in Example 2.

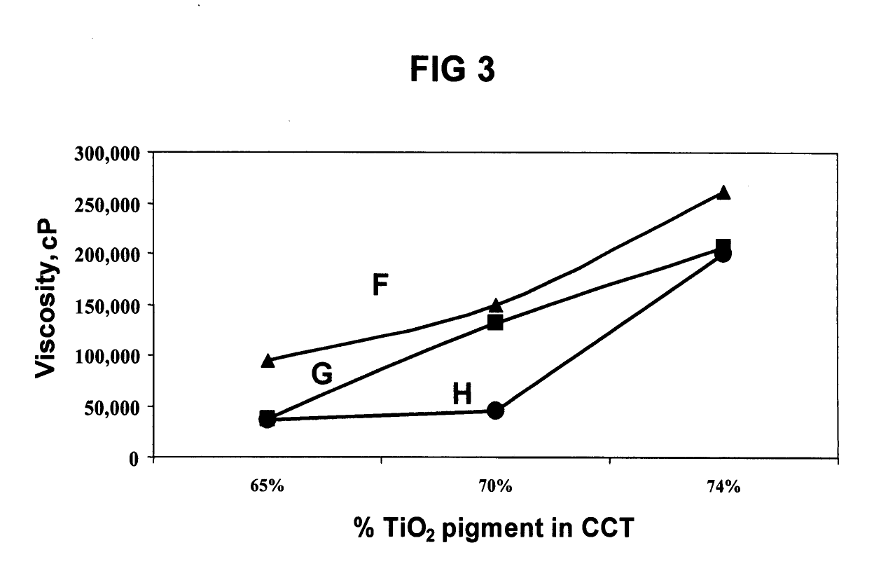

The results in FIG. 3 indicates that the viscosities of dispersions of the surface-untreated TiO₂ dispersion using composition No. 2 (curve G) and composition No. 11 (curve H) at all pigment concentrations was in fact lower than that of the surface treated TiO₂ pigment (curve F). This result again shows the unexpected superior performance of the dispersing methods and dispersing compositions of this present invention over expensive pigments whose surfaces have been chemically treated.

### Example 5

This study illustrates the effectiveness of the method and dispersing compositions of the invention in the preparation of concentrated oil dispersion of pigment mixtures. A mixture of powders of TiO₂ (85%), yellow iron oxide (9%), red iron oxide (5%), and black iron oxide (1%) is blended by the method described above to a uniform color using a Osterizer blender in place of a COWLES mixer. A concentrated dispersion, consisting of 70% of the blended pigment powders, 3.5% composition No. 2 of Table 1, and 26.5% Caprylic/Capric Triglyceride oil is easily prepared according the experimental conditions of Example 2. The pigments are virgin pigments whose surfaces are untreated. This dispersion is shiny, smooth, and flowable. It is readily rubbed over the skin with a pleasant silky and soft sensory feel. This concentrated pigment dispersion is labeled as CP-1 and is used in the formulation of finished color cosmetic products as described below.

### Example 6

This Example illustrates the application of the concentrated oil dispersion of pigments (CP-1) for the preparation of color cosmetic formulations. Table 3 lists non-limiting examples of cosmetic formulations that can be prepared with CP-1 using common laboratory mixing equipments. Formula A and B are water in oil emulsions foundations and formula C and D are oil in water emulsions foundations and skin creams. Lipsticks, eye liners among other types of products can be similarly formulated.

TABLE 2
Color cosmetics prepared with pigment dispersion of the invention

| | A | B | C | D |
|---|---|---|---|---|
| | Weight Percent in Composition | | | |
| PHASE A | | | | |
| PEG 30 Dipolyhydroxystearate | 3.0% | | | |
| Triglyceryl Diisostearate | | 4% | | |
| Steareth-2 | | | 3% | 3% |
| Steareth-21 | | | 2% | 2% |
| Isononyl Isononate | 20% | 5% | 10% | 10% |
| Octyl Palmitate | 5% | 15% | 10% | 10% |
| Polyethylene | 1.5% | 2% | 1% | 1% |
| PHASE B | | | | |
| CP-1 pigment dispersion | 10% | 10% | 10% | 6% |
| PHASE C | | | | |
| Glycerine | 5% | 3% | 3% | 3% |
| Sepigel 350 | | | 2% | 2% |
| NaCl | 1.5% | 1.5% | | |
| EDTA | 0.05% | 0.05% | 0.05% | 0.05% |
| Water | 53.15% | 58.65% | 57.32% | 62.15% |
| PHASE D | | | | |
| Preservative | 0.3% | 0.3% | 0.3% | 0.3% |
| Perfume | 0.5% | 0.5% | 0.5% | 0.5% |

### Example 7

This study further illustrates the application of the dispersing method and dispersing compositions of this invention for the preparation of concentrated oil dispersions of UV shielding particles and pigments. The compositions shown in Table 3 were prepared by the method described above.

All compositions were prepared by mixing the dispersing compositions and carrier oil, followed by gradual addition of the powders and milling with a COWLES disperser at room temperature. The dispersions could be milled further with ball mill or other high intensity milling machines. All of these concentrated dispersions were shinny white materials, flow-able under shear and spread easily on the skin.

TABLE 3
Examples of concentrated dispersions of
UV shielding particles and pigments

| | E | F | G | H |
|---|---|---|---|---|
| M262* | 55% | 55% | | |
| TiO2 pigment | | | 65% | 65% |
| Dispersing composition 10 of | 8% | | 7% | |
| Table 1 | | | | |
| Dispersing composition 7 of | | 7% | | 7% |
| Table 1 | | | | |
| C₁₂₋₁₃ Alkyl alcohol Benzoate | 37% | 38% | 28% | 28% |
| Viscosity, cP | 3300 cP | 3400 cP | 100,000 cP | 4400 cP |

*M262, supplied by Kemira Inc., ultra fine titanium dioxide powder, coated with alumina and dimethicone

### Example 8

This Example illustrates the application of the concentrated oil dispersion of UV shielding particles of this invention as an easy-to-use intermediate in the formulation of sun protection products.

The preparation method, in general, involves mixing the concentrated oil dispersions of UV shielding particles together with the oil soluble ingredients of phase A using a simple overhead mixer at room temperature or at a temperature above the melting point of the solid ingredients (other than the metal oxides), followed by the normal emulsification procedures using well known colloid mill or homogenizing equipment. These formulations have good sensory property when applied to skin and are visibly transparent on skin.

TABLE 4
Sun care formulations

| | I | J | K | L |
|---|---|---|---|---|
| Phase A | | | | |
| PEG 30 Dipolyhydroxystearate | 3.0% | 3.0% | | |
| Triglyceryl Diisostearate | 0.5% | 0.5% | | |
| Steareth-2 | | | 3% | 3% |
| Steareth-21 | | | 2% | 2% |
| Isononyl Isononate | 10% | | 5% | 3% |
| Octyl Palmitate | 10% | 10% | 2% | 5% |
| Hydrogenated Polybutene | | | 5% | 2% |
| #1 of Table 3 | 10% | | | 20% |
| #2 of Table 3 | | 15% | 10% | |
| Octinoxiate | | | 7% | |
| Phase B | | | | |
| Glycerine | 5% | 5% | 5% | 5% |
| Sepigel 350 | | | 2% | 2% |
| NaCl | 1.5% | 1.5% | | |
| EDTA | 0.05% | 0.05% | 0.05% | 0.05% |
| Water | 58.95% | 63.95% | 57.95% | 61.95% |
| Phase D | | | | |
| Preservative | 0.5% | 0.5% | 0.5% | 0.5% |
| Perfume | 0.5% | 0.5% | 0.5% | 0.5% |

While this invention has been described with respect to particular embodiments thereof, it is apparent that numerous other forms and modifications of the invention will be obvious to those skilled in the art. The appended claims and this invention generally should be construed to cover all such obvious forms and modifications which are within the true spirit and scope of the present invention.

What is claimed is:

1. A method for preparing a dispersion of pigment, metal oxide or filler particulate solids in oil, said method comprising the step of combining the particulate solids in powder form with a dispersing composition under suitable shear to form a uniform dispersion, said dispersing composition comprising:
a. 10% to 90% by weight of total composition of an oil soluble surfactant selected from the group consisting of phospholipids, alkyl acids, nonionic surfactants having an HLB less than 10, and mixtures thereof;
b. 10% to 90% by weight of total composition of either an anhydride based polymer selected from the group consisting of polyalkene succinic anhydrides; maleinized polyisoprene; maleinized polybutadiene; maleinized copolymers of isoprene and butadiene; copolymers of styrene and maleic anhydride; derivatives formed by reaction of the anhydride group of the anhydride based polymer with one or more of water, amines, alcohols, or polyols; and mixtures thereof; or a mixture of the said anhydride based polymer with polyhydroxystearic acid; and
c. 0 to 50% by weight of composition of one or more liquid oil carrier.

2. A method according to claim 1 wherein the phospholipids are selected from the group consisting of lecithin, phosphatidylcholine, phosphatidylethanolamine, phosphatidylinositol, phosphatidic acid and mixtures thereof.

3. A method according to claim 1 wherein the alkyl acid has a molecular weight less than 1200 Daltons and is an alkyl or alkyl alkoxylated carboxylic acid, a dimer acid of unsaturated fatty acids, a trimer acid of unsaturated fatty acids, an alkyl or alkyl alkoxylated phosphoric acid, alkyl or alkyl alkoxylated phosphoric acids salt or mixtures thereof.

4. A method according to claim 1 wherein the nonionic surfactant has an HLB less that 6.

5. A method according to claim 1 wherein the anhydride based polymer has a molecular weight of 1,000 to 10,000 Daltons and wherein the anhydride based polymer contains less than five anhydride groups or anhydride equivalent groups.

6. A method according to claim 1 wherein the anhydride based polymers are dermatologically acceptable.

7. A method according to claim 1 wherein 10 to 90% of the total composition is an anhydride based polymer selected from the group consisting of maleinized polybutadiene, maleinized polyisoprene, maleinized polybutadiene, maleinized polyisoprene, derivatives formed by reaction of the anhydride group of these anhydride based polymers with one or more of water, amines, alcohols, or polyols; and mixtures thereof.

8. A composition suitable for dispersing particulate materials in oil said composition comprising:
i) 10% to 90% by weight of total composition of an oil soluble surfactant selected from the group consisting of phospholipids, alkyl acids, nonionic surfactants having an HLB less than 10, and mixtures thereof;
ii) 10% to 90% by weight of total composition of either an anhydride based polymer selected from the group consisting of polyalkene succinic anhydrides; maleinized polyisoprene; maleinized polybutadiene; maleinized copolymers of isoprene and butadiene; copolymers of styrene and maleic anhydride; derivatives formed by reaction of the anhydride group of the anhydride based polymer with one or more of water, amines, alcohols, or polyols; and mixtures thereof; or a mixture of the said anhydride based polymer with polyhydroxystearic acid; and;
iii) 0 to 50% by weight of composition of a liquid oil carrier.

9. A composition according to claim 8 wherein the composition is dermatologically acceptable.

10. A concentrated dispersion comprising:
i) 50% to 80% by weight of the concentrated dispersion of pigments, fillers, metal oxides or combinations thereof in particulate form
ii) 3% to 20% by weight of pigment in the concentrated dispersion composition comprising:
a. 10% to 90% by weight of dispersing composition of an oil soluble surfactant selected from the group consisting of phospholipids, alkyl acids, nonionic surfactants having an HLB less than 10, and mixtures thereof;
b. 10% to 90% by weight of total composition of either an anhydride based polymer selected from the group consisting of polyalkene succinic anhydrides; maleinized polyisoprene; maleinized polybutadiene; maleinized copolymers of isoprene and butadiene; copolymers of styrene and maleic anhydride; derivatives formed by reaction of the anhydride group of the anhydride based polymer with one or more of water, amines, alcohols, or polyols; and mixtures thereof; or a mixture of the said anhydride based polymer with polyhydroxystearic acid; and;
iii) a liquid oil.

11. A concentrated dispersion according to claim 10, wherein the viscosity of the dispersion is less than 300,000 cP measured at 1 RPM.

12. A concentrated dispersion according to claim 10, wherein the metal oxide has a primary particle size of less than 0.2 u and is a UV screen selected from the group consisting of TiO₂, ZnO, CeO₂, BaTiO₃, SrTiO₃, and mixtures thereof.

13. A concentrated dispersion according to claim 10, wherein the pigments comprise titanium dioxide, zinc oxide, iron oxides and iron hydroxide of red, black, brown, ultramarine blue, and yellow colors, organic lakes and mixtures thereof.

14. A concentrated dispersion according to claim 13, wherein the fillers include mica, talc, sericite, calcium carbonate, kaolin, silica or mixtures thereof.

15. A health, beauty or personal care composition comprising:
i) 0.04% to 2% by weight of total composition of the anhydrous dispersing composition comprising:
a. 10% to 90% by weight of dispersing composition of an oil soluble surfactant selected from the group consisting of phospholipids, alkyl acids, nonionic surfactants having an HLB less than 10, and mixtures thereof;
b. 10% to 90% by weight of dispersing composition of either an anhydride based polymer selected from the group consisting of polyalkene succinic anhydrides; maleinized polyisoprene; maleinized polybutadiene; maleinized copolymers of isoprene and butadiene; copolymers of styrene and maleic anhydride; derivatives formed by reaction of the anhydride group of the anhydride based polymer with one or more of water, amines, alcohols, or polyols; and mixtures thereof; or a mixture of the said anhydride based polymer with polyhydroxystearic acid;
c. 0 to 50% by weight of the dispersing composition of a liquid oil carrier;
ii) 1% to 30% of a dermatologically acceptable metal oxide, pigment, filler or mixtures thereof; and
iii) one or more ingredients selected from the group consisting of additional liquid oil carrier, active ingredients, benefit agents, and auxiliary ingredients.

16. A health, beauty or personal care composition according to claim 15, wherein the anhydrous composition is present at a level of from 0.15% to 0.6% and the metal oxide is present at a level of from 5% to 10%.

17. A health, beauty or personal care composition according to claim 15, wherein the metal oxide has a primary particle size of less than 0.2 u and is a UV screen selected from the group consisting of TiO₂, ZnO, CeO₂, BaTiO₃, SrTiO₃, and mixtures thereof.

18. A health, beauty or personal care composition according to claim 15, wherein the one or more ingredients comprise skin moisturizers, skin whitening agents, vitamins, anti-oxidants, blood clotting agents, wound healing agents, absorbents for wounds, antibacterial, hair conditioner, UV filters, insect repellants, anti-aging agents, emulsifiers, emollients, fragrance, colorants, preservatives, and other suitable carriers.

19. An anhydrous composition suitable for dispersing particulate materials in oils, said composition comprising:
i) 10% to 60% by weight of composition of polyhydroxystearic acid;
ii) 10% to 60% by weight of composition of an oil soluble surfactants selected from synthetic or natural phospholipids, dimer acids or trimer acids; and
iii) 0% to 50% of a liquid oil carrier.

20. A concentrated dispersion comprising
i) 50% to 80% by weight of concentrated dispersion of a metal oxide, pigment, filler or combination thereof in particulate form
ii) 3% to 20% by weight of pigment of a concentrated dispersion of an anhydrous dispersing composition comprising:
a. 10% to 60% by weight of dispersing composition of polyhydroxystearic acid;
b. 10% to 60% by weight of dispersing composition of an oil soluble surfactants selected from synthetic or natural phospholipids, dimmer acids or trimer acids; and
iii) a liquid oil.
